(19) United States
(12) Patent Application Publication
(10) Pub. No.: US 2025/0100970 A1
(43) Pub. Date: Mar. 27, 2025

(54) THERAPEUTIC COMPOUNDS AND METHODS OF USE THEREOF

(71) Applicant: Tessellate Therapeutics Inc., Henderson, NV (US)

(72) Inventors: Dustin Hines, Henderson, NV (US); Rochelle Hines, Henderson, NV (US)

(21) Appl. No.: 18/730,135

(22) PCT Filed: Jan. 18, 2023
(86) PCT No.: PCT/US23/60844

§ 371 Date: Jul. 18, 2024

Related U.S. Application Data
(60) Provisional application No. 63/300,604, filed on Jan. 18, 2022.

(51) Int. Cl. C07D 213/36; A61K 31/137; A61K 31/341; A61K 31/381; A61K 31/4418; C07C 217/60; C07D 233/64; C07D 263/32; C07D 277/28; C07D 307/52; C07D 333/20

(52) U.S. Cl. CPC C07D 213/36; A61K 31/137; A61K 31/341; A61K 31/381; A61K 31/4418; C07C 217/60; C07D 233/64; C07D 263/32; C07D 277/28; C07D 307/52; C07D 333/20; C07C 2601/16

(57) ABSTRACT

Provided are compounds of Formulae (I), (II) and (III) and therapeutic use of the compounds:

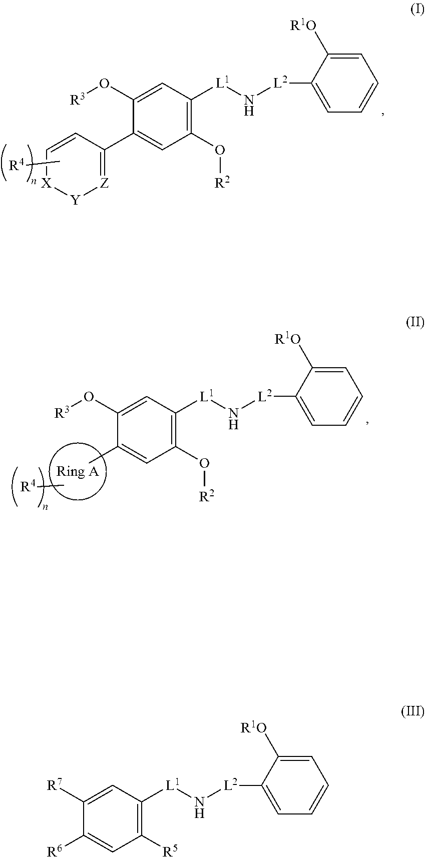

### CROSS-REFERENCES TO RELATED APPLICATIONS

This application claims the benefit of priority of U.S. Provisional Application No. 63/300,604 filed on Jan. 18, 2022, which is incorporated herein by reference in its entirety and for all purposes.

### BACKGROUND

Depression is a highly prevalent mental health illness. Treatment resistant depression is particularly problematic. It would be desirable to have new therapeutic agents, including agents to treat depression.

### SUMMARY OF THE INVENTION

We now provide new compounds and methods for treatment using such compounds.

In a first aspect, compounds of Formula (T) are provided:

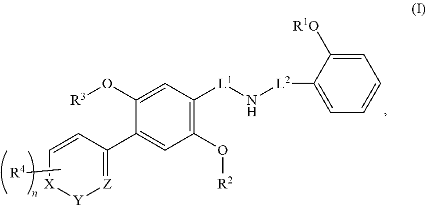

or pharmaceutically acceptable salt thereof,
wherein:
X, Y, and Z are each independently —C═ or —N═
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

In embodiments, in Formula (I), X is —C═, Y is —C═; and Z is —C═.

In certain embodiments, in Formula (I), X is —N═, Y is —C═ and Z is —C═.

In certain embodiments, in Formula (I), X is —N═, Y is —N═ and Z is —C═.

In certain embodiments, in Formula (I), X is —N═, Y is —C═ and Z is —N═.

In certain embodiments, in Formula (I), L¹ and LU are each independently substituted or unsubstituted C₁-C₄ alkylene.

In certain embodiments, in Formula (I), R¹, R², and R³ are each independently hydrogen, or substituted or unsubstituted C₁-C₄ alkyl.

In certain embodiments, in Formula (I), R¹ is hydrogen, and R² and R³ are each independently substituted or unsubstituted C₁-C₄ alkyl.

In certain embodiments, n is 0.

Exemplary preferred compounds of Formula (I) include the following compounds:

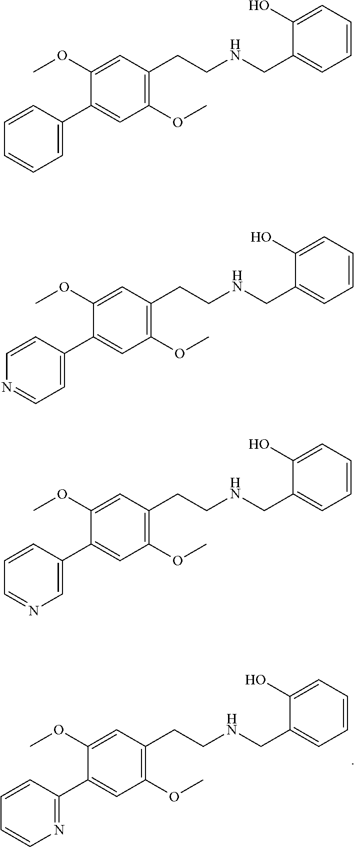 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O]

In a second aspect, compounds of Formula (II) are provided:

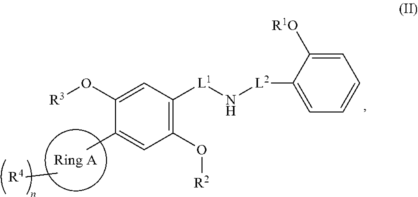

or pharmaceutically acceptable salt thereof,
wherein:
Ring A is substituted or unsubstituted 5 membered heteroaryl or heterocyclic ring containing O, S, or N;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

In certain embodiments, in Formula(II), Ring A is

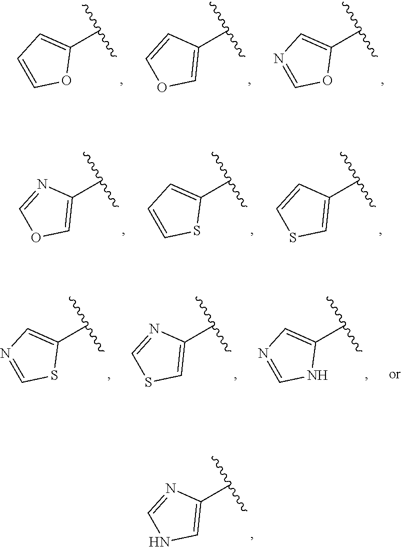

wherein the squiggly line (“ [symbol] ”) is the attachment point to the phenyl ring.

In certain embodiments, in Formula (II), L¹ and L² are each independently substituted or unsubstituted C₁-C₄ alkylene.

In certain embodiments, in Formula (II), R¹, R², and R³ are each independently hydrogen, or substituted or unsubstituted C₁-C₄ alkyl.

In certain embodiments, in Formula (II), R¹ is hydrogen, and R² and R³ are each independently substituted or unsubstituted C₁-C₄ alkyl.

In certain embodiments, in Formula (II), n is (0).

In certain embodiments, in Formula (II), R² is substituted or unsubstituted C₁-C₄ alkyl.

Exemplary preferred compounds of Formula (II) include the following compounds:

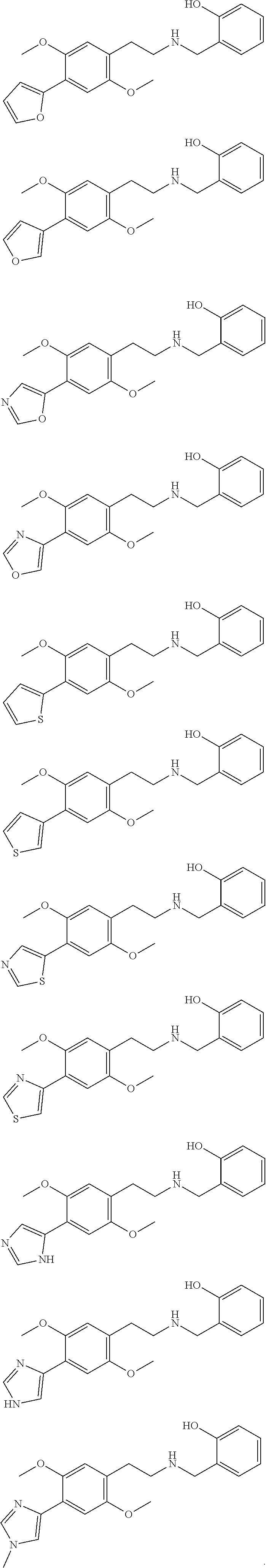

In a third aspect, compounds of Formula (III) are provided:

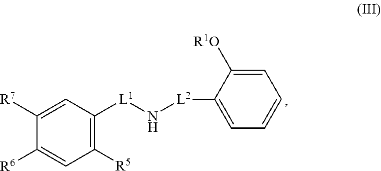

or pharmaceutically acceptable salt thereof,
wherein:
L¹ and L² are each independently a bond or substituted or unsubstituted alkylene;
R¹ is hydrogen, or substituted or unsubstituted alkyl,
R⁵ and R⁷ are each independently hydrogen, halogen, substituted or unsubstituted alkyl, or substituted or unsubstituted heteroalkyl;
R⁶ is substituted or unsubstituted phenyl, or substituted or unsubstituted 5 membered heteroaryl or heterocyclic ring containing O, S, or N.

In certain embodiments, in Formula (II), L¹ and L² are each independently substituted or unsubstituted C₁-C₄ alkylene.

In certain embodiments, in Formula (III), R¹ is hydrogen, or substituted or unsubstituted C₁-C₄ alkyl.

In certain embodiments, R⁵ and R⁷ are each independently hydrogen, halogen, substituted or unsubstituted C₁-C₄ alkyl, or substituted or unsubstituted 2 to 4 membered heteroalkyl. In certain embodiments, R⁵ and R⁷ are each independently hydrogen, —Cl, C₇-C₄ haloalkyl, or C₁-C₄ alkoxyl.

In certain embodiments, R⁶ is substituted or unsubstituted phenyl, substituted or unsubstituted pyridyl, or substituted or unsubstituted 5 membered heteroaryl or heterocyclic ring selected from

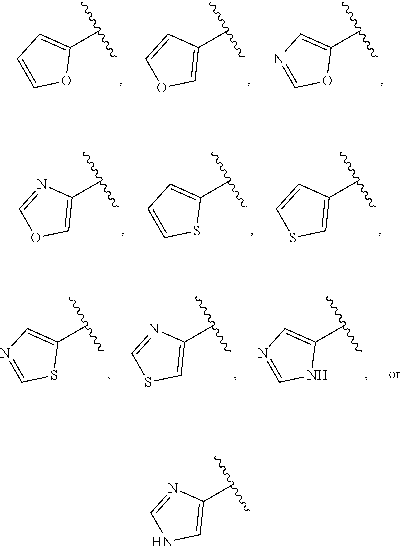

wherein the squiggly line (“ [symbol] ”) is the attachment point to the phenyl ring.

Exemplary preferred compounds of Formula (III) include the following compounds:

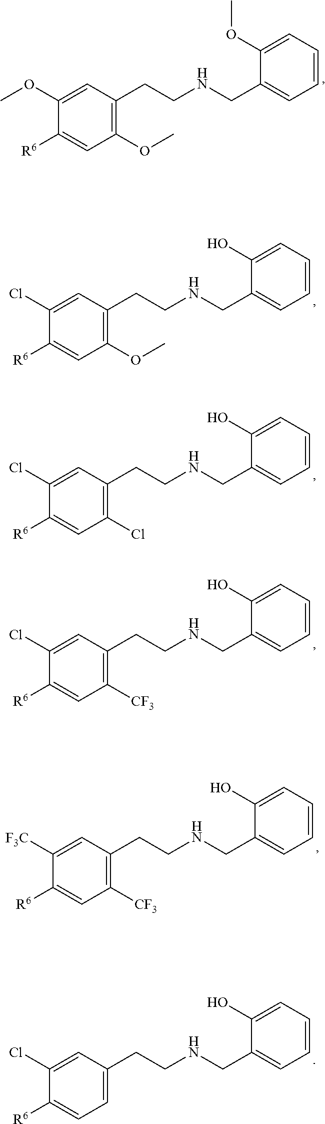

The present compounds of Formulae (I), (II) and/or (III) can be useful to are useful for treatment of a number of disorders.

In one aspect, a compound of Formulae (I), (II) and/or (III) may be used to treat a subject suffering from or susceptible to treatment resistant depression.

In one aspect, a compound of Formulae (I), (II) and/or (III) may be used to treat a subject suffering from or susceptible to post-traumatic stress disorder.

In one aspect, a compound of Formulae (I), (II) and/or (III) may be used to treat a subject suffering from or susceptible to substance use disorder.

In one aspect, a compound of Formulae (I), (II) and/or (III) may be used to treat a subject suffering from or susceptible to psychiatric disorder related sleep disturbances.

In one aspect, a compound of Formulae (I), (II) and/or (III) may be used to treat a subject suffering from or susceptible to a disorder or disease related to dysregulated synaptic plasticity.

In one aspect, a compound of Formulae (I), (II) and/or (III) may be used to treat a subject suffering from or susceptible to a disease or disorder related to dysregulated synaptogenesis.

In accordance with the present invention, a compound of Formulae (I), (II) and/or (III) may be compounds may be formulated into pharmaceutical compositions suitable for oral (immediate release and modified-release), parenteral, topical, rectal, vaginal, intranasal, inhalation or liquid administration, such as oral dosage forms like pills (e.g., tablets, capsules, caplets, troaches, lozenges, caches, gelcaps, caps, pellets, boluses, pastilles, orally disintegrating tablets, sublingual tablets and buccal tablets), thin films, powders, granules, crystals, liquid solutions, syrups, emulsions and suspensions, topical dosage forms like pastes, creams, ointments, gels, liquids, sprays, skin patches, dermal patches, balms, salves and implants, ophthalmic and otic dosage forms, e.g., drops, suspensions, emulsions, creams and gels, vaginal rings and inserts, suppositories, inhalation dosage forms like aerosols, inhalers, nebulizers and vaporizers, and parenteral dosage forms like intradermal (ID), intramuscular (IM), intraosseous (IO), intraperitoneal (IP), intravenous (IV), caudal, intrathecal (ITH), subcutaneous (SC), and the like.

Other aspects of the invention are disclosed infra.

### DETAILED DESCRIPTION

Serotonergic hallucinogens act as agonists at serotonin receptors (5-HTRs), and are known for their mind-altering qualities, which are best described as alterations in mood, perception, and other cognitive faculties (Aghajanian, & Marek, Neuropsychopharmacology 1999; 21:16-23; Vollenweider, Vollenweider-Scherpenhuyzen, Bäbler, Vogel, & Hell. Neuroreport 1998; 9:3897-3902). The mind-altering qualities make hallucinogens an attractive agent for a variety of purposes, including a long history of ritualistic practices, and more recently as rapidly acting treatments for psychiatric disorders. Study of these drugs dropped sharply in the 1960s and 1970s due to the illicit stigma associated with these drugs tied to their recreational use. Recently however, they have reemerged as therapies for the psychological distress associated with terminal illnesses such as cancer (Reiche, Hermle, Gutwinski, Jungaberle, Gasser, Majić. Prog Neuropsychopharmacology Biol Psychiatry 2018; 81:1-10). Following these studies, recent research suggests that psilocybin is effective for treatment resistant depression (Carhart-Harris. Bolstridge, Day, Rucker, Watts, Erritzoe, Kaelen, Giribaldi, Bloomfield, Pilling, et al. Psychopharmacology, 2018; 235:399-408) and may outperform selective serotonin reuptake inhibitors (Carhart-Harris, Giribaldi, Watts., Baker-Jones, Murphy-Beiner, Murphy, Martell, Blemings, Erritzoe. Nutt. N Engl J Med. 2021 384(15):1402-1411), the current gold standard in depression treatment. Psilocybin is also suggested to be effective in treatment of other psychiatric disorders including substance use disorder (Bogenschutz, Curr. Drug Abuse Rev. 2013; 6:17-29), and post-traumatic stress disorder (Bird, Modlin, Rucker. Int Rev Psychiatry. 2021 33(3):229-249).

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein as a shorthand notation to represent the expression “and,” describing the combination of items, as well as “or,” describing the items in the alternative form.

As used herein, the term “treatment-resistant” means a lack of therapeutic response after at least one trial of an antidepressant at an adequate dose for six weeks.

The term “compound” when referring to a compound of this invention, refers to a collection or population of molecules having an identical chemical structure, except that there may be isotopic variation among the constituent atoms of the molecules.

Certain compounds of the present invention possess asymmetric carbon atoms (optical or chiral centers) or double bonds: the enantiomers, racemates, diastereomers, tautomers, geometric isomers, stereoisometric forms that may be defined, in terms of absolute stereochemistry, as (R)-or (S)-isomers, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

The term “about”, as used herein, means an acceptable margin of error for a particular value, which depends in part on how the value is measured or determined. In certain embodiments, “about” as used herein will be understood by persons of ordinary skill in the art to mean up to plus or minus 20% of the particular term. In further embodiments, “about” as used herein will be understood by persons of ordinary skill in the art to mean up to plus or minus 10% of the particular term.

The term “isolated” as used herein, means having been removed from or is free of other compounds or biological and/or chemical materials present when the compound is first formed. The term “isolated” embraces compounds isolated from natural sources as well as chemically-synthesized compounds.

As used herein, the term “substantially pure” means sufficiently homogeneous to appear free of readily detectable impurities as determined by standard analytical methods, such as thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” refers to a collection of molecules, wherein at least about 55%, about 60%, about 65%, about 70%, about 75%, about 80%, about 85%, about 90%, about 95%, about 97%, about 98%, about 98.5%, about 99%, about 99.5% or about 99.9% or greater of the molecules are a single compound, including a racemic mixture or a single stereoisomer thereof, as determined by standard analytical methods.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting; includes allowing a compound described herein to interact with a protein or enzyme that is involved in a signaling pathway.

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a, reduction in the activity of a, particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

As used herein, and unless otherwise specified, the terms “treat,” “treating” and “treatment” refer to the eradication or amelioration of a disease, disorder, or condition, or of one or more symptoms associated with the disease, disorder or condition. In certain embodiments, the terms refer to minimizing the advancement or worsening of the disease, disorder, or condition resulting from the administration of a formulation of the invention to a patient with such a disease, disorder, or condition. In some embodiments, the terms refer to the administration of a formulation provided herein, after the onset of symptoms of the particular disease, disorder, or condition. The terms “treat,” “treating” “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition. In a particular embodiment the terms “treat,” “treating”, “treatment”, or the like, covers the treatment of a disease, disorder, or condition in a mammal, e.g. a primate, e.g., a human, and includes at least one of (i), (ii), and (iii) above. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the terms “subject”, and “patient” are used interchangeably. The terms “subject” and “patient” refer to an animal (e.g., a bird such as a chicken, quail or turkey) or a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

As used herein, and unless otherwise specified, the terms “prevent,” “preventing” and/or “prevention” refer to the prevention of the onset, recurrence or spread of a disease, disorder, or condition, or of one or more symptoms thereof. In certain embodiments, the terms refer to the administration of a compound of any one of Formulae (I), (IT) and/or (III), or a pharmaceutically acceptable salt thereof, to a subject, with or without other additional active compounds, prior to the onset of symptoms, particularly to patients at risk of a disease, disorder, or condition provided herein. The terms encompass the inhibition or reduction of a symptom of the particular disease, disorder, or condition. Subjects with familial history of a disease, disorder, or condition, in particular, are candidates for preventive regimens in certain embodiments. In addition, subjects who have a history of recurring symptoms are also potential candidates for the prevention. In this regard, the terms “prevent,” “preventing” and/or “prevention” may be interchangeably used with the term “prophylactic treatment.”

As used herein, and unless otherwise specified, a “therapeutically effective amount” or an “effective amount” of an active agent of a compound of any one of Formulae (I), (TT) and/or (TIT) or a pharmaceutically acceptable salt thereof, is an amount sufficient to provide a therapeutic benefit in the treatment or management of a disease, disorder, or condition, or to delay or minimize one or more symptoms associated with the disease, disorder, or condition The term “therapeutically effective amount” can encompass an amount that improves overall therapy, reduces or avoids symptoms or causes of disease, disorder, or condition, or enhances the therapeutic efficacy of another therapeutic agent. The therapeutically effective amount for a particular patient in need of such treatment can be determined by considering various factors, such as the condition treated, the overall health of the patient, method of administration, the severity of side-effects, and the like. For any compound described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

As used herein, and unless otherwise specified, the terms “manage,” “managing” and “management”, are used interchangeably herein and refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the terms “manage,” “managing” and/or “management”, encompass treating a subject who had suffered from the particular disease, disorder, or condition in an attempt to prevent or minimize the recurrence of the disease, disorder, or condition.

As used herein, and unless otherwise specified, a “prophylactically effective amount” of a compound of any one of Formulae (I), (II) and/or (III) is an amount sufficient to prevent a disease, disorder, or condition, or prevent its recurrence.

“Patient” or “subject” or “subject in need thereof” refers to a living organism or animal diagnosed with, suffering from or prone to a disease, disorder or condition that can be treated by administration of a compound as disclosed herein. Non-limiting examples include humans, other mammals, bovines, rats, mice, dogs, monkeys, goat, sheep, cows, deer, and other non-mammalian animals. In some embodiments, a patient is human.

### Compounds

Compounds having therapeutic effects (e.g., methods for treatment) are provided.

In an aspect, provided is a compound of Formula (I):

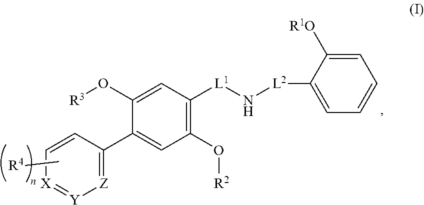

or pharmaceutically acceptable salt thereof
wherein:
X, Y, and Z are each independently —C═ or —N═;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl and
n is an integer of 0 to 5.

In embodiments, X is —C═. In embodiments, X is N. In embodiments, Y is —C═. In embodiments, Y is —N═. In embodiments, Z is —C═. In embodiments, Z is —N═.

In embodiments, X is —C═, Y is —C═ and Z is —C═. In embodiments, X is —N═, Y is —C═ and Z is —C═. In embodiments, X is —N═, Y is —N═ and Z is —C═. In embodiments, X is —N═Y is —C═ and Z is —N═.

In certain embodiments, the compound has the structure of Formula (IA):

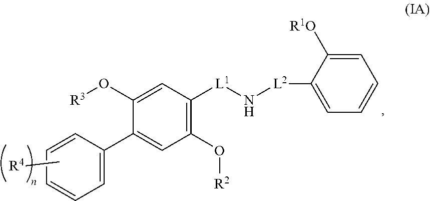

or pharmaceutically acceptable salt thereof.

In certain embodiments, the compound has the structure of Formula (IB):

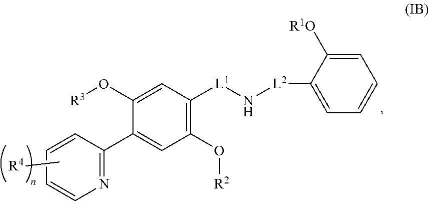

or pharmaceutically acceptable salt thereof.

In certain embodiments, the compound has the structure of Formula (IC):

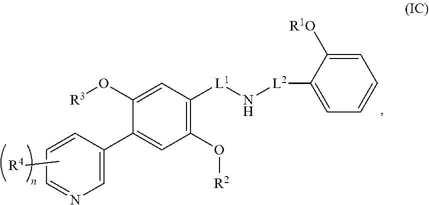

or pharmaceutically acceptable salt thereof.

In certain embodiments, the compound has the structure of Formula (ID):

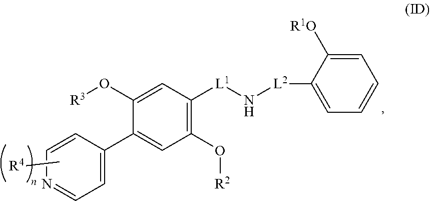

or pharmaceutically acceptable salt thereof.

In embodiments, L¹ and L² are each independently substituted or unsubstituted C₁-C₄ alkylene (e.g., methylene, ethylene, propylene, isopropylene, butylene, or t-butylene).

In embodiments, L¹ is substituted or unsubstituted C₁-C₄ alkylene (e.g., methylene, ethylene, propylene, isopropylene, butylene, or t-butylene). In embodiments, L¹ is substituted C₁-C₄ alkylene (e.g. methylene, ethylene, propylene, isopropylene, butylene, ort-butylene). In embodiments, L is unsubstituted C₁-C₄ alkylene (e.g., methylene, ethylene, propylene, isopropylene, butylene, or t-butylene), In embodiments, L¹ is methylene. In embodiments, L¹ is ethylene. In embodiments, L¹ is propylene. In embodiments, L¹ is isopropylene. In embodiments, L¹ is butylene. In embodiments, L¹ is t-butylene.

In embodiments, L² is substituted or unsubstituted C₁-C₄ alkylene (e.g., methylene, ethylene, propylene, isopropylene, butylene, or t-butylene). In embodiments. LC is substituted C₁-C₄ alkylene (e.g., methylene, ethylene, propylene, isopropylene, butylene, or t-butylene). In embodiments, L² is unsubstituted C₁-C₄ alkylene (e.g., methylene, ethylene, propylene, isopropylene, butylene, or t-butylene). In embodiments, L² is methylene. In embodiments, L² is ethylene. In embodiments, L² is propylene. In embodiments, L² is isopropylene. In embodiments, L² is butylene. In embodiments, L² is t-butylene.

In certain embodiments, L¹ is methylene and L² is ethylene. In certain embodiments, L¹ is ethylene and L² is methylene.

In embodiments, R¹, R², and R³ are each independently hydrogen, or substituted or unsubstituted C₁-C₄ alkyl (e.g., methyl, ethyl, propyl, isopropyl, butyl, or t-butyl).

In embodiments, R¹ is hydrogen. In embodiments. R¹ is substituted C₁-C₄ alkyl (e.g., methyl, ethyl, propyl, isopropyl, butyl, or t-butyl). In embodiments, R¹ is methyl. In embodiments, R¹ is ethyl. In embodiments, R¹ is propyl. In embodiments, R¹ is isopropyl. In embodiments, R¹ is butyl. In embodiments, R¹ is t-butyl.

In embodiments, R² is hydrogen. In embodiments. R² is substituted C₁-C₄ alkyl (e.g., methyl, ethyl, propyl, isopropyl, butyl, or t-butyl). In embodiments, R² is methyl.

In embodiments, R¹ is ethyl. In embodiments, R² is propyl. In embodiments. R² is isopropyl. In embodiments, R² is butyl. In embodiments, R² is t-butyl.

In embodiments, R³ is hydrogen. In embodiments, R³ is substituted C₁-C₄ alkyl (e.g., methyl, ethyl, propyl, isopropyl, butyl, ort-butyl). In embodiments, R³ is methyl. In embodiments, R³ is ethyl. In embodiments, R³ is propyl. In embodiments, R³ is isopropyl. In embodiments, R³ is butyl. In embodiments, R³ is t-butyl.

In certain embodiments. R¹ is hydrogen and R² and R³ are methyl.

In certain embodiments, n is 0. In certain embodiments, n is 1. In certain embodiments, n is 2. In certain embodiments, n is 3.

In embodiments, a compound of Formula (I) is:

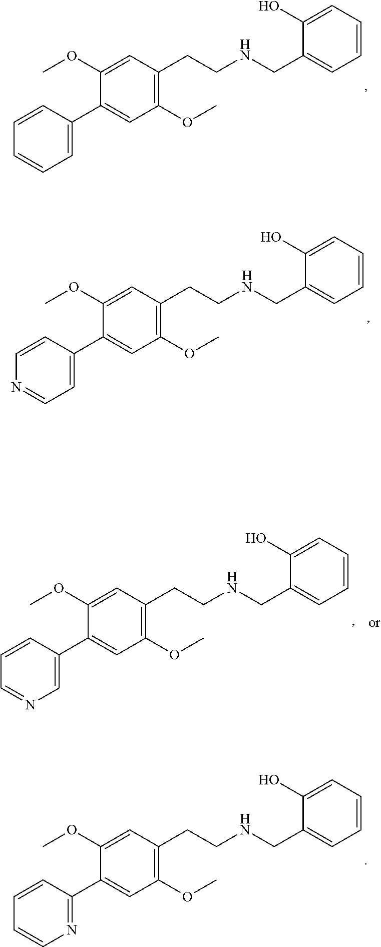 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O]

In an aspect, provided is a compound of Formula (II):

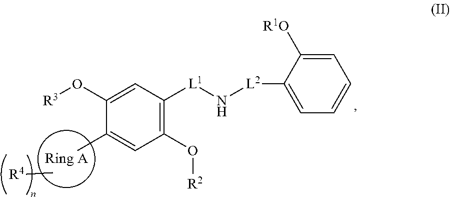

or pharmaceutically acceptable salt thereof,
wherein:
Ring A is substituted or unsubstituted 5 membered heteroaryl or heterocyclic ring containing O, S, or N;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and n is an integer of 0 to 5.

In embodiments, Ring A

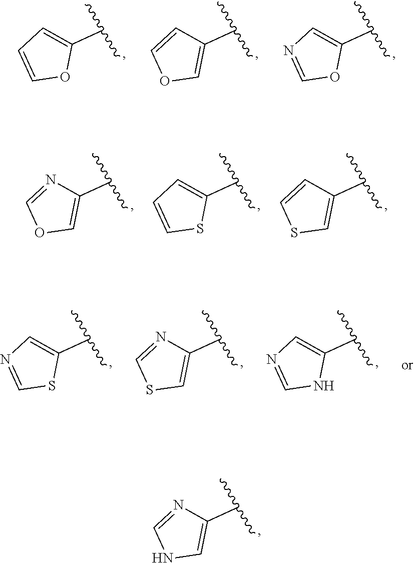

wherein the squiggly line (“ [symbol] ”) is the attachment point to the phenyl ring.

In embodiments, L and L are each independently substituted or unsubstituted C₁-C₄ alkylene (e.g., methylene, ethylene, propylene, isopropylene, butylene, or t-butylene).

In embodiments, L¹ is substituted or unsubstituted C₁-C₄ alkylene (e.g., methylene, ethylene, propylene, isopropylene, butylene, or t-butylene). In embodiments, L¹ is substituted C₁-C₄ alkylene (e.g., methylene, ethylene, propylene, isopropylene, butylene, ort-butylene). In embodiments, L is unsubstituted C₁-C₄ alkylene (e.g., methylene, ethylene, propylene, isopropylene, butylene, or t-butylene). In embodiments, L¹ is methylene. In embodiments, L¹ is ethylene. In embodiments, L¹ is propylene. In embodiments, L¹ is isopropylene. In embodiments, L¹ is butylene.

In embodiments, L¹ is t-butylene.

In embodiments, L² is substituted or unsubstituted C₁-C₄ alkylene (e.g., methylene, ethylene, propylene, isopropylene, butylene, or t-butylene). In embodiments, L² is substituted C₁-C₁ alkylene (e.g., methylene, ethylene, propylene, isopropylene, butylene, or t-butylene). In embodiments, L² is unsubstituted C₁-C₄ alkylene (e.g., methylene, ethylene, propylene, isopropylene, butylene, or t-butylene). In embodiments, L² is methylene. In embodiments, L² is ethylene. In embodiments, L² is propylene. In embodiments, L² is isopropylene. In embodiments, L² is butylene. In embodiments, L² is t-butylene.

In certain embodiments, L¹ is methylene and L² is ethylene. In certain embodiments, L¹ is ethylene and L² is methylene.

In embodiments, R¹, R², and R³ are each independently hydrogen, or substituted or unsubstituted C₁-C₄ alkyl (e.g., methyl, ethyl, propyl, isopropyl, butyl, or t-butyl).

In embodiments, R¹ is hydrogen. In embodiments, R¹ is substituted C₁-C₄ alkyl (e.g., methyl, ethyl, propyl, isopropyl, butyl, or t-butyl). In embodiments, R¹ is methyl. In embodiments, R¹ is ethyl. In embodiments, R¹ is propyl. In embodiments, R¹ is isopropyl. In embodiments, R¹ is butyl. In embodiments, R¹ is t-butyl.

In embodiments, R² is hydrogen. In embodiments, R² is substituted C₁-C₄ alkyl (e.g. methyl, ethyl, propyl, isopropyl, butyl, or t-butyl). In embodiments, R, is methyl. In embodiments, R² is ethyl. In embodiments, R² is propyl. In embodiments. R² is isopropyl. In embodiments, R² is butyl. In embodiments, R² is t-butyl.

In embodiments, R³ is hydrogen. In embodiments, R³ is substituted C₁-C₄ alkyl (e.g., methyl, ethyl, propyl, isopropyl, butyl, or t-butyl). In embodiments, R³ is methyl. In embodiments, R³ is ethyl. In embodiments, R³ is propyl. In embodiments, R³ is isopropyl. In embodiments, R³ is butyl. In embodiments, R³ is t-butyl.

In certain embodiments, R¹ is hydrogen and R² and R³ are methyl.

In certain embodiments, n is 0. In certain embodiments, n is 1.

In embodiments, a compound of Formula (II) is:

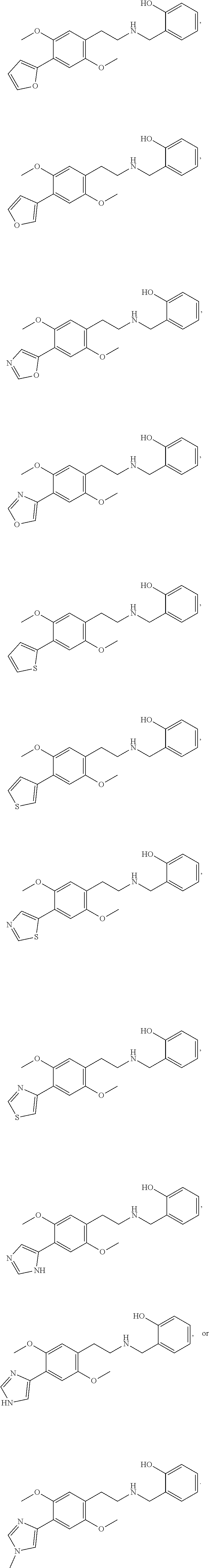

In an aspect, provided is a compound of Formula (III):

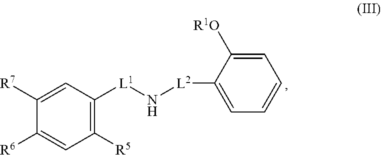

Or pharmaceutically acceptable salt thereof,
wherein:
L¹ and L² are each independently a bond or substituted or unsubstituted alkylene;
R¹ is hydrogen, or substituted or unsubstituted alkyl:
R⁵ and R⁷ are each independently hydrogen, halogen, substituted or unsubstituted alkyl, or substituted or unsubstituted heteroalkyl;
R⁶ is substituted or unsubstituted phenyl, or substituted or unsubstituted 5 membered heteroaryl or heterocylic ring containing O, S, or N.

In embodiments, L¹ and L² are each independently substituted or unsubstituted C₁-C₄ alkylene (e.g., methylene, ethylene, propylene, isopropylene, butylene, or t-butylene).

In embodiments, L¹ is substituted or unsubstituted C₁-C₄ alkylene (e.g., methylene, ethylene, propylene, isopropylene, butylene, or t-butylene). In embodiments, L¹ is substituted C₁-C₄ alkylene (e.g., methylene, ethylene, propylene, isopropylene, butylene, ort-butylene). In embodiments, L is unsubstituted C₁-C₄ alkylene (e.g., methylene, ethylene, propylene, isopropylene, butylene, or t-butylene). In embodiments, L¹ is methylene. In embodiments, L¹ is ethylene. In embodiments, L¹ is propylene. In embodiments, L¹ is isopropylene. In embodiments, L is butylene. In embodiments, L¹ is t-butylene.

In embodiments, L² is substituted or unsubstituted C₁-C₄ alkylene (e.g., methylene, ethylene, propylene, isopropylene, butylene, or t-butylene). In embodiments, L² s substituted C₁-C₄ alkylene (e.g., methylene, ethylene, propylene, isopropylene, butylene, or t-butylene). In embodiments, L² is unsubstituted C₁-C₄ alkylene (e.g., methylene, ethylene, propylene, isopropylene, butylene, or t-butylene). In embodiments, L² is methylene. In embodiments, L² is ethylene. In embodiments, L² is propylene. In embodiments, L² is isopropylene. In embodiments, L² is butylene. In embodiments, L² is t-butylene.

In certain embodiments, L¹ is methylene and L² is ethylene. In certain embodiments, L is ethylene and L² is methylene.

In embodiments, R¹ is hydrogen. In embodiments, R¹ is substituted C₁-C₄ alkyl (e.g., methyl, ethyl, propyl, isopropyl, butyl, or t-butyl). In embodiments, R¹ is methyl. In embodiments, R¹ is ethyl. In embodiments, R¹ is propyl. In embodiments, R¹ is isopropyl. In embodiments, R¹ is butyl. In embodiments, R¹ is t-butyl.

In embodiments, R⁵ and R⁷ are each independently hydrogen, halogen, substituted or unsubstituted C₁-C₄ alkyl, or substituted or unsubstituted 2 to 4 membered heteroalkyl.

In embodiments, R⁵ is hydrogen. In embodiments, R⁵ is halogen (e.g., F, Cl, Br, or I). In embodiments, R⁵ is substituted or unsubstituted C₁-C₄ alkyl (e.g., methyl, ethyl, propyl, isopropyl, butyl, ort-butyl). In embodiments, R⁵ is substituted C₁-C₄ alkyl (e.g., haloalkyl). In embodiments, R⁵ is unsubstituted C₁-C₄ alkyl (e.g., haloalkyl). In embodiments, R⁵ is substituted or unsubstituted 2 to 4 membered heteroalkyl. In embodiments, R⁵ is —OR^{5A}, and R^{5A} is unsubstituted C₁-C₄ alkyl.

In embodiments, R⁷ is hydrogen. In embodiments, R⁷ is halogen (e.g., F, Cl, Br, or I). In embodiments, R⁷ is substituted or unsubstituted C₁-C₄ alkyl (e.g., methyl, ethyl, propyl, isopropyl, butyl, or t-butyl). In embodiments, R⁷ is substituted C₁-C₄ alkyl (e.g., haloalkyl). In embodiments, R⁷ is unsubstituted C₁-C₄ alkyl (e.g., haloalkyl). In embodiments, R⁷ is substituted or unsubstituted 2 to 4 membered heteroalkyl. In embodiments, R⁷ is —OR⁷ is —OR^{7A}, and R^{7A} is unsubstituted C₁-C₄ alkyl.

In embodiments, R⁶ is substituted or unsubstituted phenyl. In embodiments, R⁶ is substituted or unsubstituted 5 membered heteroaryl or heterocyclic ring containing O, S, or N. In embodiments, P is substituted 5 membered heteroaryl or heterocyclic ring containing O, S. or N. In embodiments, R⁶ is unsubstituted 5 membered heteroaryl or heterocyclic ring containing O, S or N.

In embodiments, R⁶ is substituted or unsubstituted 5 membered heteroaryl containing O. In embodiments, R⁶ is substituted or unsubstituted 5 membered heteroaryl ring containing S. In embodiments, R⁶ is substituted or unsubstituted 5 membered heteroaryl ring containing N.

In embodiments, R⁶ is substituted or unsubstituted 5 membered heterocyclic ring containing O. In embodiments, R⁶ is substituted or unsubstituted 5 membered heterocyclic ring containing S. In embodiments, R⁶ is substituted or unsubstituted 5 membered heterocyclic ring containing N.

In embodiments, a compound of Formula (III) is:

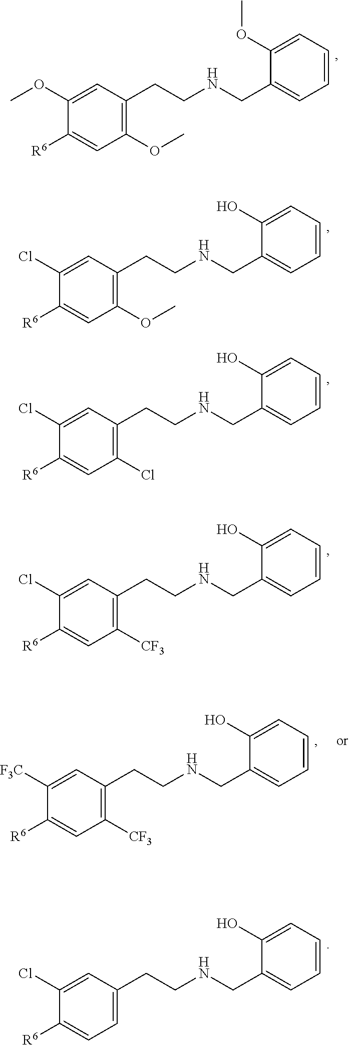

### Pharmaceutical Compositions and Formulations

In as aspect, provided are compositions (e.g. pharmaceutical composition) for use of treating a subject. The compositions may include one or more compounds having a structure of Formula (I), (II), and/or (III, or pharmaceutically acceptable salt thereof, and pharmaceutically acceptable carriers or excipients.

The pharmaceutical composition may be suitably formulated for co-administration. The “co-administer” is meant that a composition described herein is administered at the same time, just prior to, or just after the administration of one or more additional therapies. The compounds provided herein can be administered alone or can be co-administered to the patient. Coadministration is meant to include simultaneous or sequential administration of the compounds individually or in combination (more than one compound). Thus, the preparations can also be combined, when desired, with other active substances (e.g. to reduce metabolic inhibition).

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached, Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

In certain embodiments, the pharmaceutical composition is formulated for intranasal or inhalation, like aerosols, inhalers, nebulizers and vaporizers, administration.

In certain embodiments, the pharmaceutical composition is formulated for intraoral administration.

In certain embodiments, the pharmaceutical composition is formulated for parenteral administration, such as intravenous, intramuscular, intradermal, subcutaneous, intraosseous, caudal, intrathecal or intraperitoneal administration.

In certain embodiments, the pharmaceutical composition is formulated for administration by infusion.

In certain embodiments, the pharmaceutical composition is formulated for sublingual, orally disintegrating or buccal administration.

In certain embodiments, the pharmaceutical composition is formulated for ophthalmic or optic administration.

In certain embodiments, the pharmaceutical composition is formulated for topical administration like pastes, creams, ointments, gels, liquids, sprays, skin patches, dermal patches, balms, salves and implants.

In certain embodiments of the present invention, the pharmaceutical composition (e.g., a pharmaceutical composition formulated for, for example, oral (e.g., intraoral administration or a tablet, caplet, gelcap, cap or capsule composition), rectal, vaginal, intransal, inhalation, otic, ophthalmic, topical, sublingual, orally disintegation, buccal, parenteral, intravenous, subcutaneous or intramuscular administration, or formulated for administration by infusion, comprises an amount of a compound of any one of Formulae (I), (II) or (III).

### EXAMPLE

### Example 1: Synthesis

The present compounds can be readily including for example by reaction of a primary amine with a substituted alkyl halide. For instance, cesium hydroxide can be used in a coupling reaction which can promote a selective N-monoalkylation of primary amines to prepare various secondary amines efficiently.

Compounds (1A-1 to 2A-2) are synthesized according to following Scheme 1.

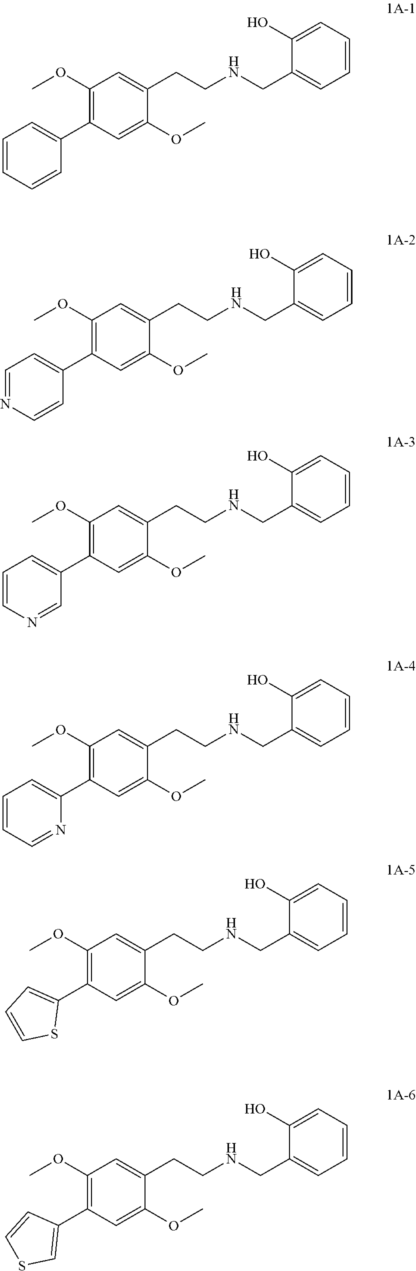 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O ; COc1cc(-c2cccs2)c(OC)cc1CCNCc1ccccc1O ; COc1cc(-c2ccsc2)c(OC)cc1CCNCc1ccccc1O]

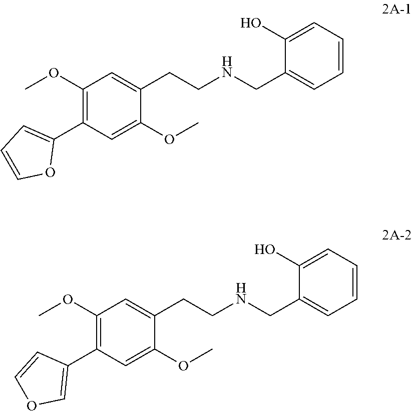 [SMILES: COc1cc(-c2ccco2)c(OC)cc1CCNCc1ccccc1O ; COc1cc(-c2ccoc2)c(OC)cc1CCNCc1ccccc1O]

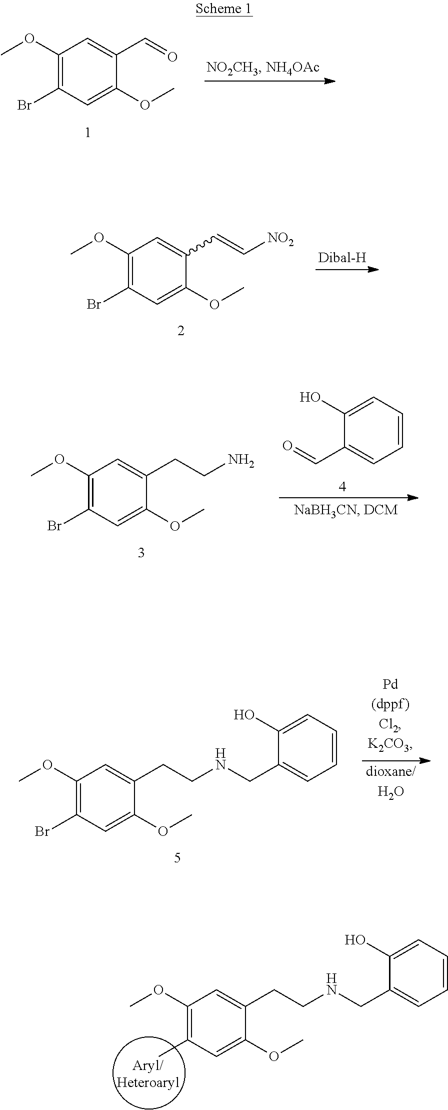

### Step 1

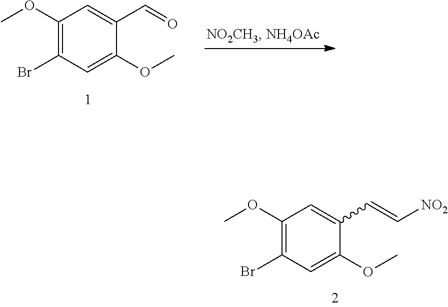

To a solution of 4-bromo-2,5-dimethoxybenzaldehyde (1, 5.0 g, 20.4 mmol) in MeNO₂ (50 mL), was added NH₄O Ac (1.58 g, 20.4 mmol) and the reaction mixture was stirred at reflux for 1.5 h. After cooling down, excess MeNO₂ was removed in vacuo and the residue was dissolved in EA (100 mL). The organic layer was washed with water, dried over MgSO₄, filtered and evaporated in vacuo. The residue was purified by flash chromatography (PE/EA=9:1) to give the title compound 1-bromo-2,5-dimethoxy-4-(2-nitrovinyl)benzene (2, 4.2 g, purity:85%) as a yellow solid.

### Step 2

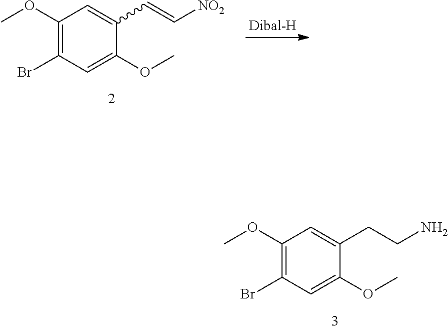 [SMILES: COc1cc(C=C[N+](=O)[O-])c(OC)cc1Br ; COc1cc(CCN)c(OC)cc1Br]

To a solution of 1-bromo-2,5-dimethoxy-4-(2-nitrovinyl)benzene (2) (4.2 g, 14.58 mmol) in THF (20 mL) was dropwise a solution of Dibal-H (102 mL, 102 mmol, 1M in THF) at 0° C., then the mixture was heated to 60° C. for 2 hours. After cooling down, NH₄Cl solution added, extracted with EA, dried over MgSO₄, filtered and concentrated to get the crude product 2-(4-bromo-2,5-dimethoxyphenyl)ethanamine (3, etude), which was used directly at next step without further purification.

### Step 3

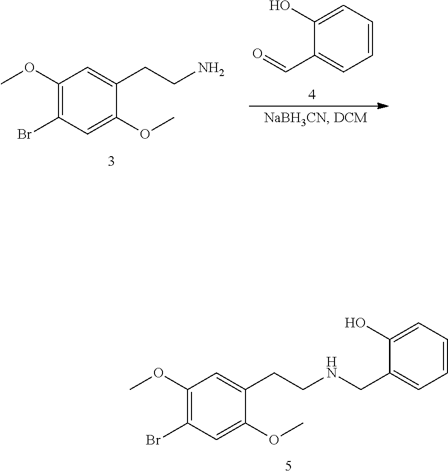 [SMILES: COc1cc(CCN)c(OC)cc1Br ; O=Cc1ccccc1O ; COc1cc(CCNCc2ccccc2O)c(OC)cc1Br]

A mixture of 2-(4-bromo-2,5-dimethoxyphenyl)ethanamine (3, crude) and 2-hydroxybenzaldehyde (4, 1.5 g, 12.4 mmol) in DCM (40 mL) was added NaBH₃CN (1.6 g, 24.5 mmol) in portions and one drop AcOH, then the mixture was stirred overnight at it. NaHCO₃ solution added, extracted with DCM, dried over MgSO₄, filtered and evaporated in vacuo. The residue was purified by flash chromatography (PE/EA=2:1) to give the title compound 2-(((4-bromo-2,5-dimethoxyphenethyl)amino)methyl)phenol (5, 1.6 g, yield:32%) as a colorless oil.

### Step 4

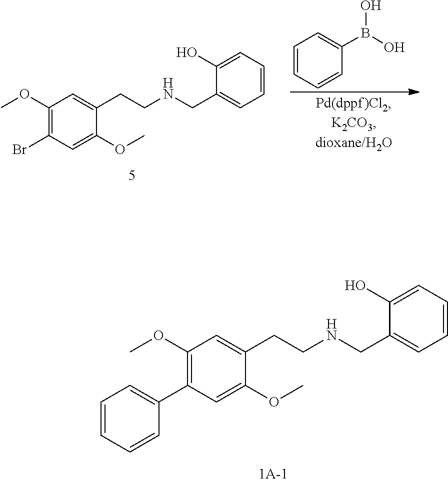

A mixture of 2-(((4-bromo-2,5-dimethoxyphenethyl)amino)methyl)phenol (5, 400 mg, 1.0 mmol) phenylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2-(2,5-dimethoxy-[1,1′-biphenyl]-4-yl)ethyl)amino)methyl)phenol (1A-1, 56 mg, yield: 15%) as a brown solid.

### Step 5

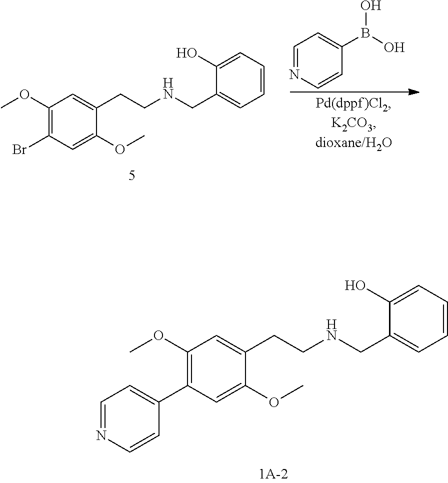

A mixture of 2-(((4-bromo-2,5-dimethoxyphenethyl)amino)methyl)phenol (5, 400 mg, 1.0 mmol), pyridin-4-ylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-4-yl)phenethyl)amino)methyl)phenol (1A-2, 120 mg, yield:26%) as a yellow solid.

### Step 6

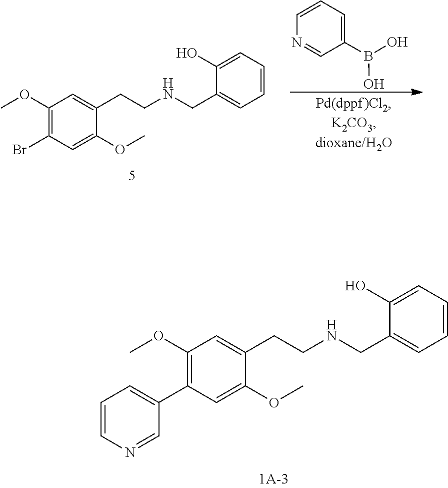 [SMILES: COc1cc(CCNCc2ccccc2O)c(OC)cc1Br ; OB(O)c1cccnc1 ; COc1cc(-c2cccnc2)c(OC)cc1CCNCc1ccccc1O]

A mixture of 2-(((4-bromo-2,5-dimethoxyphenethyl)amino)methyl)phenol (5, 400 mg, 1.0 mmol), pyridin-3-ylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄C₁ solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-3-yl)phenethyl)amino)methyl)phenol (1A-3, 70 mg, yield: 15%) as a yellow solid.

### Step 7

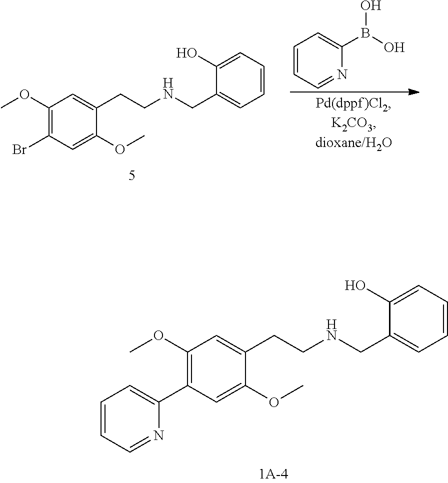

A mixture of 2-(((4-bromo-2,5-dimethoxyphenethyl)amino)methyl)phenol (5, 500 mg, 1.3 mmol), pyridin-2-ylboronic acid (400 mg, 3.2 mmol), K₂CO₃ (415 mg, 10 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-2-yl)phenethyl)amino)methyl)phenol (1A-4, 110 tug, yield:18%) as a yellow solid.

### Step 7

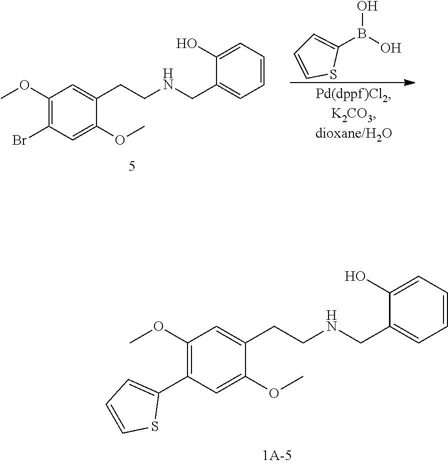

A mixture of 2-(((4-bromo-2,5-dimethoxyphenethyl)amino)methyl)phenol (5, 400 mg, 1.0 mmol), thiophen-2-ylboronic acid (300 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA:==2:1) and Prep-HPLC to give the tile compound 2-(((2,5-dimethoxy-4-(thiophen-2-yl)phenethyl)amino)methyl)phenol (1A-5, 120 mg, yield:26%) as a white solid.

### Step 8

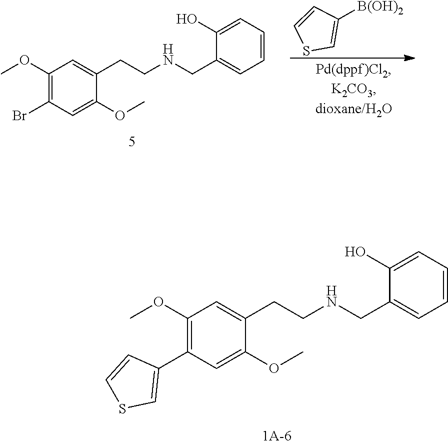

A mixture of 2-(((4-bromo-2,5-dimethoxyphenethyl)amino)methyl)phenol (5, 400 mg, 1.0 mmol), thiophen-3-ylboronic acid (300 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried Over MgSO₄, filtered and evaporated in vacuo. The residue was purified by flash chromatography (PE/EA=2:1) and Prep-HPLC to give the tile compound 2-(((2,5-dimethoxy-4-(thiophen-3-yl)phenethyl)amino)methyl)phenol (1A-6, 110 mg, yield:25%) as a white solid

### Step 9

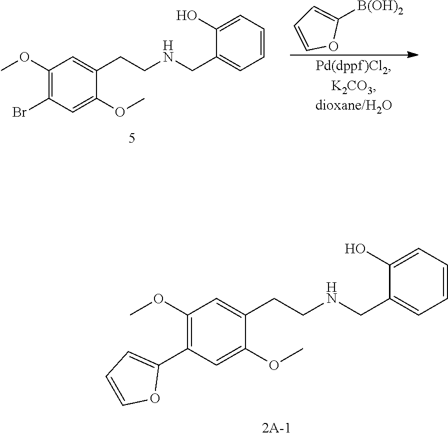

A mixture of 2-(((4-bromo-2,5-dimethoxyphenethyl)amino)methyl)phenol (5, 400 mg, 1.0 mmol), furan-2-ylboronic acid (180 mg, 1.6 mmol), K₂CO₃ (415 mg, 3.0 mmol) and Pd(dppf)(Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA:=2:1) and Prep-HPLC to give the tile compound 2-(((4-(furan-2-yl)-2,5-dimethoxyphenethyl)amino)methyl)phenol (2A-1, 120 mg, yield:24%) as a white solid.

### Step 10

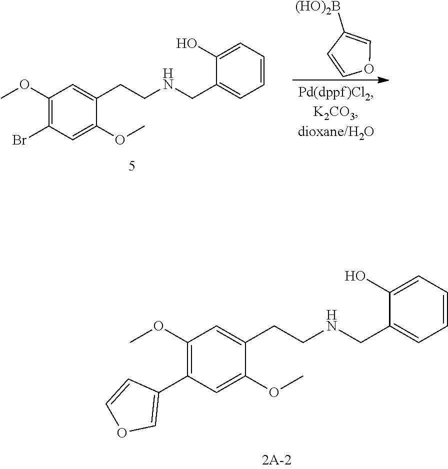 [SMILES: COc1cc(CCNCc2ccccc2O)c(OC)cc1Br ; OB(O)c1ccoc1 ; COc1cc(-c2ccoc2)c(OC)cc1CCNCc1ccccc1O]

A mixture of 2-(((4-bromo-2,5-dimethoxyphenethyl)amino)methyl)phenol (5, 400 mg. 1.0 mmol), furan-3-ylboronic acid (180 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA:=2:1) and Prep-1-HPLC to give the tile compound 2-(((4-(furan-2-yl)-2,5-dimethoxyphenethyl)amino)methyl)phenol (2A-2, 58 mg, yield:16%) as a brown solid.

The invention claimed is:

1. A compound of Formula (I): 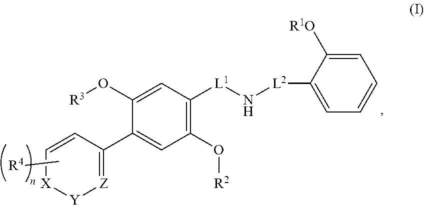
or pharmaceutically acceptable salt thereof,
wherein:
X, Y, and Z are each independently —C═ or —N═;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

2. A compound of claim 1 wherein the compound is:

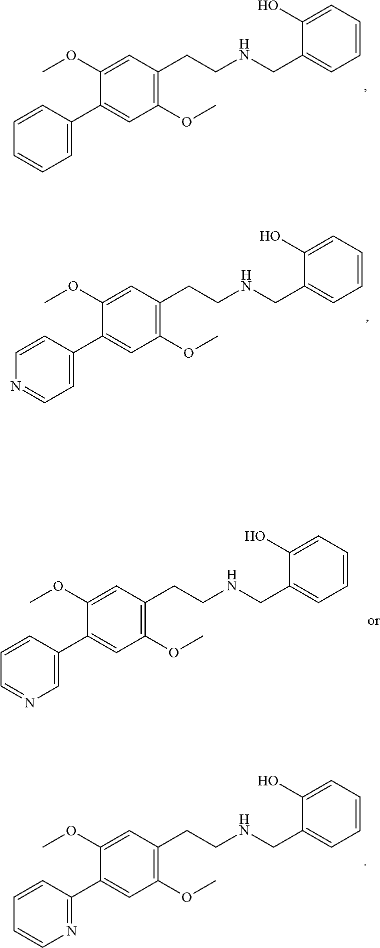 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O]

3. A compound of Formula (II): 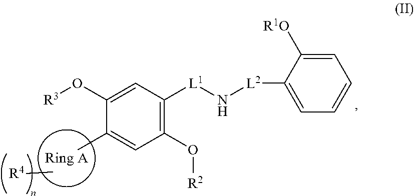
or pharmaceutically acceptable salt thereof,
wherein:
Ring A is substituted or unsubstituted 5 membered heteroaryl or heterocyclic ring containing O, S, or N;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

4. A compound of claim 3 wherein Formula(II), Ring A is 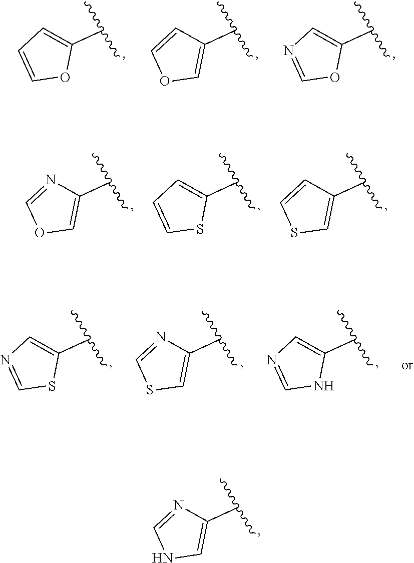
wherein the squiggly line (“ [symbol] ”) is the attachment point to the phenyl ring.

5. A compound of claim 3 wherein the compound is:

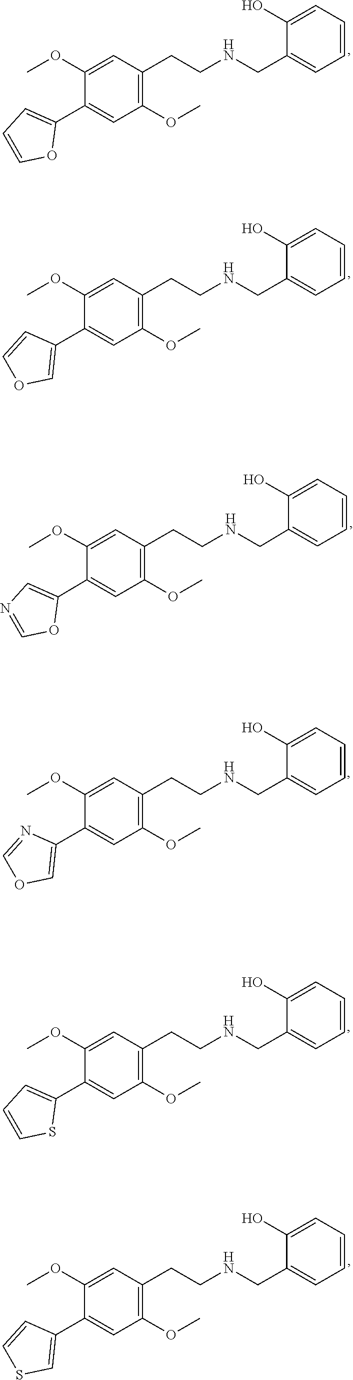 [SMILES: COc1cc(-c2ccco2)c(OC)cc1CCNCc1ccccc1O ; COc1cc(-c2ccoc2)c(OC)cc1CCNCc1ccccc1O ; COc1cc(-c2cnco2)c(OC)cc1CCNCc1ccccc1O ; COc1cc(-c2cocn2)c(OC)cc1CCNCc1ccccc1O ; COc1cc(-c2cccs2)c(OC)cc1CCNCc1ccccc1O ; COc1cc(-c2ccsc2)c(OC)cc1CCNCc1ccccc1O]

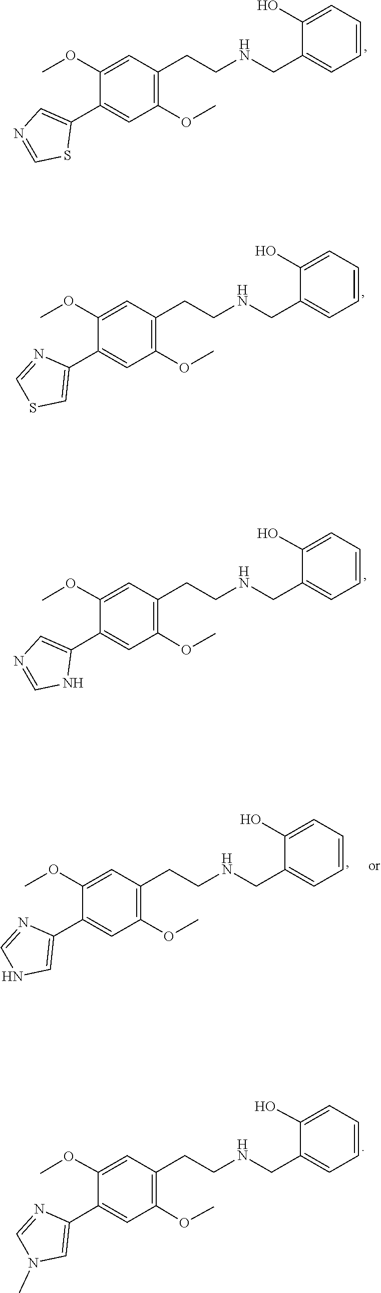 [SMILES: COc1cc(-c2cncs2)c(OC)cc1CCNCc1ccccc1O ; COc1cc(-c2cscn2)c(OC)cc1CCNCc1ccccc1O ; COc1cc(-c2cnc[nH]2)c(OC)cc1CCNCc1ccccc1O ; COc1cc(-c2c[nH]cn2)c(OC)cc1CCNCc1ccccc1O ; COc1cc(-c2cn(C)cn2)c(OC)cc1CCNCc1ccccc1O]

6. A compound of the following Formula (III): 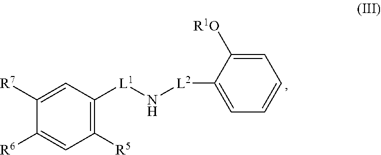
or pharmaceutically acceptable salt thereof,
wherein:
L¹ and L² are each independently a bond or substituted or unsubstituted alkylene;
R¹ is hydrogen, or substituted or unsubstituted alkyl;
R⁵ and R⁷ are each independently hydrogen, halogen, substituted or unsubstituted alkyl, or substituted or unsubstituted heteroalkyl;
R⁶ is substituted or unsubstituted phenyl, or substituted or unsubstituted 5 membered heteroaryl or heterocyclic ring containing O, S, or N.

7. A compound of claim 6 that is:

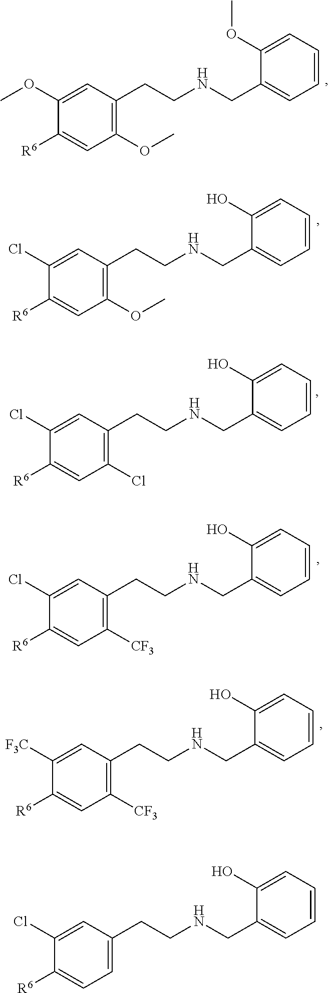

8. A pharmaceutical composition comprising a pharmaceutically acceptable carrier and one or more compounds of claim 1.

9. A method of treating a subject suffering from or susceptible to treatment-resistant depression, comprising administering to the subject an effective amount of claim 1.

10. A method of treating a subject suffering from or susceptible to post-traumatic stress disorder, comprising administering to the subject an effective amount of a compound of claim 1.

11. A method of treating a subject suffering from or susceptible to substance use disorder, comprising administering to the subject an effective amount of a compound of claim 1.

12. A method of treating a subject suffering from or susceptible to psychiatric disorder related sleep disturbances, comprising administering to the subject an effective amount of a compound of claim 1.

13. A method of treating a subject suffering from or susceptible to psychiatric disorder related to a disorder or disease related to dysregulated synaptic plasticity, comprising administering to the subject an effective amount of a compound of claim 1.

14. A method of treating a subject suffering from or susceptible to a disease or disorder related to dysregulated synaptogenesis, comprising administering to the subject an effective amount of a compound of claim 1.
